(19) United States
(12) Patent Application Publication
(10) Pub. No.: US 2005/0175867 A1
(43) Pub. Date: Aug. 11, 2005

(54) Room-temperature molten salt, process for producing the same and applications thereof

(75) Inventors: Kenji Adachi, Tsukuba-shi (JP); Kuroki Yoshichika, Tsukuba-shi (JP); Yuuko Sakamaki, Tsukuba-shi (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: DAIKIN INDUSTRIES, LTD, Osaka 530-8323 (JP)

(21) Appl. No.: 10/516,296

(22) PCT Filed: Jun. 13, 2003
(86) PCT No.: PCT/JP03/07529

(30) Foreign Application Priority Data
Jun. 18, 2002 (JP) 2002-177036
Feb. 4, 2003 (JP) 2003-27251

(51) Int. Cl. C07D 213/18; C07D 233/66

(52) U.S. Cl. 429/12; 546/347; 548/338.1

(57) ABSTRACT

The present invention provides a room-temperature molten salt that is obtainable by mixing two or more organic salts and that has a solidifying point lower than the solidifying point (or melting point) of any of the starting organic salts, a process for producing the same, and use of the same. Specifically, the present invention provides a room-temperature molten salt that comprises a mixture of two or more organic salts with different anionic moieties and different organic cationic moieties and that has a melting point lower than any of the individual organic salts, a process for producing the same, and use of the same.

### TECHNICAL FIELD

The present invention relates to a room-temperature molten salt obtainable by mixing two or more organic salts, a process for producing the same, and use of the same.

### BACKGROUND ART

Room-temperature molten salts have relatively high electric conductivity, a wide potential window and unique characteristics not possessed by conventional electrolyte systems, i.e., nonflammability and nonvolatility. Thus, researchers have been studying the possibility of using room-temperature molten salts as battery electrolytes. Moreover, room-temperature molten salts have high polarity and can dissolve a variety of organic and inorganic compounds, and therefore room-temperature molten salts are being studied as environmentally friendly “green” solvents to be used in organic and inorganic reactions, catalytic reactions, biochemical reactions, liquid-liquid extraction and separation, electrochemistry and other fields.

However, many room-temperature molten salts have a relatively high melting point, and while are liquid at room temperature, are solidified with a decrease in temperature. To expand their applications, organic salts with a much lower melting point are demanded.

Generally, the synthesis process for a room-temperature molten salt comprises two steps. As shown in the following reaction scheme, the first step is a quaternarization reaction, and the subsequent second step is anion exchange. For example, the process may comprise the steps of reacting an imidazole derivative with an alkyl halide (R^{d}X) to form an imidazolium salt, and then exchanging its anion for an anion (Y⁻) that has an appropriate capability for forming a molten salt.

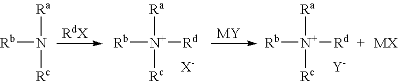

However, since room-temperature molten salts are liquid but nonvolatile, they cannot be distilled and have problems with purification. For example, in order to efficiently separate the salt (MX), which is a by-product in the process of the above reaction scheme, the following methods were proposed: a method using an expensive silver salt (J. Chem. Soc., Chem. Commun. (1992), 96); a method using the difference in solubilities (Japanese Unexamined Patent Publication No. 1996-259543); and methods comprising the step of neutralizing a tertiary amine with an organic acid to give an onium salt by protonation (Electrochem. Acta, 45, 1291 (2000); J. Electrochem. Soc., 147, 4168 (2000); Electrochem. Solid-State Lett., 4, E25 (2001); etc.). The methods using salt exchange or solubility differences are disadvantageous in view of cost and efficiency. The method of synthesizing a protonated onium salt is easy and simple, but the protonated onium salt has lower performance than alkylated onium salts.

### DISCLOSURE OF THE INVENTION

An object of the present invention is to provide a room-temperature molten salt that is obtainable by mixing two or more organic salts and that has a solidifying point lower than the solidifying point (or melting point) of any of the individual organic salts, a process for producing the same, and use of the same.

The present inventors conducted extensive research to solve the above problems and found that the above object can be achieved by mixing two or more specific organic salts. By expanding this finding, the inventors accomplished the present invention.

The present invention provides the following items:
1. A room-temperature molten salt comprising a mixture of two or more organic salts with different anionic moieties and different organic cationic moieties, the room-temperature molten salt having a solidifying point lower than that of any of the individual organic salts.
2. A room-temperature molten salt according to item 1, wherein the two or more organic salts are selected from the group consisting of the organic salts represented by formulae (I), (II), (III) and (IV): 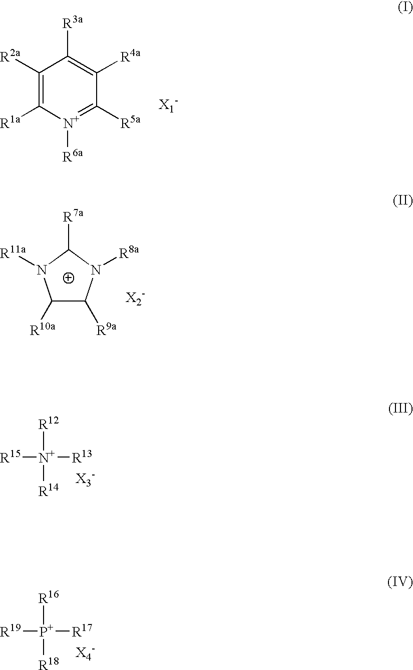
   wherein R^{1a} to R^{5a}, R^{7a}, R^{9a} and R^{10a} are the same or different and each represents a hydrogen atom, a halogen atom, an alkyl group, a cycloalkyl group, a heterocyclic group, a haloalkyl group, an aralkyl group, an aryl group, an alkoxy group, an aryloxy group or an aralkyloxy group; R^{8a} is a hydrogen atom, an alkyl group, a cycloalkyl group, a heterocyclic group, a haloalkyl group, an aralkyl group or an aryl group; R^{6a}, R^{11a}, R¹², R¹³, R¹⁴, R¹⁵, R¹⁶, R¹⁷, R¹⁸ and R¹⁹ are the same or different and each represents an alkyl group, a cycloalkyl group, a heterocyclic group, a haloalkyl group, an aralkyl group or an aryl group; two groups selected from R¹², R¹³, R¹⁴ and R¹⁵ may be linked at their ends to form, together with the adjacent nitrogen atom, a nitrogen-containing aliphatic heterocycle; two groups selected from R¹⁶, R¹⁷, R¹⁸ and R¹⁹ may be linked at their ends to form, together with the adjacent phosphorus atom, a phosphorus-containing aliphatic heterocycle; and X₁⁻, X₂⁻, X₃⁻ and X₄⁻ are each a conjugate base of a Brönsted acid.
3. A room-temperature molten salt according to item 1 or 2, wherein at least one of the two or more organic salts is a solid at room temperature.
4. A room-temperature molten salt according to item 1 or 2, wherein all of the two or more organic salts are solids at room temperature.
5. A room-temperature molten salt according to item 1 or 2, wherein at least one of the two or more organic salts is selected from the group consisting of the organic salts represented by formulae (V) and (VI): 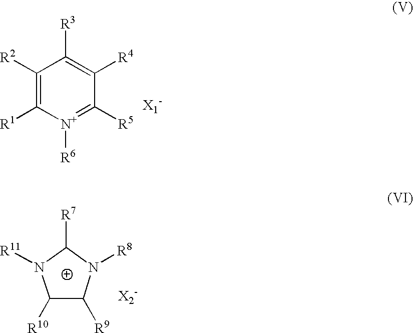
   wherein R¹ to R⁵, R⁷, R⁹ and R¹⁰ are the same or different and each represents a hydrogen atom, a halogen atom, an alkyl group, a cycloalkyl group, a heterocyclic group, a haloalkyl group, an aralkyl group, an aryl group, an alkoxy group, an aryloxy group or an aralkyloxy group; R⁸ is a hydrogen atom, an alkyl group, a cycloalkyl group, a heterocyclic group, a haloalkyl group, an aralkyl group or an aryl group; R⁶ and R¹¹ are the same or different and each represents a C₁₋₁₀ alkyl group in which at least one hydrogen atom is substituted by fluorine; and X₁⁻ and X₂⁻ are each a conjugate base of a Brönsted acid.
6. A room-temperature molten salt according to item 5, wherein all of the two or more organic salts are selected from the group consisting of the organic salts represented by formulae (V) and (VI).
7. A room-temperature molten salt according to item 5 or 6, wherein at least one of the two or more organic salts is a solid at room temperature.
8. A room-temperature molten salt according to item 5 or 6, wherein all of the two or more organic salts are solids at room temperature.
9. A room-temperature molten salt according to any one of items 5 to 8, wherein, in formulae (V) and (VI), R¹ to R⁵, R⁷, R⁹ and R¹⁰ are the same or different and each represents a hydrogen atom, a halogen atom, an alkyl group or a haloalkyl group; R⁸ is an alkyl group; R⁶ and R¹¹ are the same or different and each represents a group of the formula —CH₂R¹² wherein R¹² is a straight- or branched-chain C₁₋₉ alkyl group in which at least one hydrogen atom is substituted by fluorine.
10. A room-temperature molten salt according to item 6, wherein all of the two or more organic salts are selected from the group consisting of the organic salts represented by formula (V) and are solids at room temperature.
11. A room-temperature molten salt according to item 6, wherein all of the two or more organic salts are selected from the group consisting of the organic salts represented by formula (VI) and are solids at room temperature.
12. A room-temperature molten salt according to item 6, wherein the two or more organic salts are at least one organic salt that is selected from the group consisting of the organic salts represented by formula (V) and is a solid at room temperature, and at least one organic salt that is selected from the group consisting of the organic salts represented by formula (VI) and is solid at room temperature.
13. A room-temperature molten salt according to item 6, wherein the two or more organic salts are two organic salts that are selected from the group consisting of the organic salts represented by formulae (V) and (VI) and that are solids at room temperature; one of the organic salts having an anionic moiety represented by the formula
   (RfSO₂)₂N⁻ or (RfSO₂)(Rf′SO₂)N⁻
   wherein Rf and Rf′ are different and each represents a polyfluoroalkyl group; and
   the other of the organic salts having an anionic moiety represented by the formula
   Rf″SO₃⁻
   wherein Rf″ is a polyfluoroalkyl group.
14. A room-temperature molten salt obtainable by mixing two or more organic salts with different anionic moieties and different organic cationic moieties, the room-temperature molten salt having a solidifying point lower than that of any of the individual organic salts.
15. A process for producing a room-temperature molten salt, comprising mixing two or more organic salts with different anionic moieties and different organic cationic moieties, the room-temperature molten salt having a solidifying point lower than that of any of the individual organic salts.
16. A process according to item 15, wherein the two or more organic salts are selected from the group consisting of the organic salts represented by formulae (I) to (IV).
17. A process according to item 15 or 16, wherein at least one of the two or more organic salts is a solid at room temperature.
18. A process according to item 15 or 16, wherein all of the two or more organic salts are solids at room temperature.
19. A process according to item 15, wherein the two or more organic salts are selected from the group consisting of the organic salts represented by formulae (V) and (VI) and are solids at room temperature.
20. An electrolytic solution comprising a room-temperature molten salt according to any one of items 1 to 14.
21. A battery comprising an electrolytic solution according to item 20, a positive electrode, a negative electrode and a separator.
22. A battery according to item 21, which is a nonaqueous lithium secondary battery.
23. A solvent for use in organic reaction solvent comprising a room-temperature molten salt according to any one of items 1 to 14.
24. An extraction solvent comprising a room-temperature molten salt according to any one of items 1 to 14.
25. A capacitor comprising an electrolyte or electrolytic solution that comprises a room-temperature molten salt according to any one of items 1 to 14.
26. An electric double layer capacitor comprising an electrolyte or electrolytic solution that comprises a room-temperature molten salt according to any one of items 1 to 14.
27. A dye-sensitized solar cell comprising a room-temperature molten salt according to any one of items 1 to 14.
28. A fuel cell comprising a room-temperature molten salt according to any one of items 1 to 14.
29. A polymer electrolyte fuel cell comprising a room-temperature molten salt according to any one of items 1 to 14.

The present invention is described below in detail.

Room-temperature Molten Salt of the Present Invention

With respect to the room-temperature molten salt of the present invention, “room temperature” is in the range of from about 20° C. to about 30° C. In the present invention, an “organic salt that is a solid at room temperature” means an organic salt that is in a solid state in the temperature range of about 20° C. to about 30° C., and “room-temperature molten salt” means an organic salt that is in a liquid state in the temperature range of about 20° C. to about 30° C. The above-mentioned temperatures are all at atmospheric pressure.

The room-temperature molten salt of the present invention is produced by mixing two or more organic salts that are different from each other both in anionic moiety and cationic moiety. The room-temperature molten salt exhibits a much lower solidifying point than the solidifying points (or melting points) of the individual starting organic salts, and is obtained as a mixed organic salt that is liquid at room temperature. Namely, the room-temperature molten salt is characterized in that the salt is produced by mixing two or more organic salts with different anionic moieties and different cationic moieties, and thereby has a much lowered solidifying point than the individual organic salts.

As used herein, “anionic moiety” means a negatively charged component that constitutes a part of the each organic salt, and “cationic moiety” means a positively charged component that constitutes a part of each organic salt. As mentioned hereinafter, cationic moieties are organic.

Specifically, the room-temperature molten salt of the present invention can be obtained by mixing two or more organic salts selected from the group consisting of the organic salts represented by formulae (I), (II), (III) and (IV). In particular, to obtain the room-temperature molten salt of the present invention in high purity, it is preferable that at least one of the two or more starting organic salts is a solid at room temperature, and more preferably, all of the individual organic salts are solids at room temperature.

It is preferable that at least one, and more preferably all of the two or more starting organic salts are selected from the group consisting of the organic salts represented by formulae (V) and (VI). Also in this case, at least one of the two or more organic salts is preferably a solid at room temperature, and more preferably all of the organic salts are solids at room temperature.

Further, the room-temperature molten salt of the present invention can be obtained by mixing two or more organic salts selected from the group consisting of the organic salts represented by formula (V). Of the two or more starting organic salts, preferably at least one, and more preferably all are solids at room temperature.

Furthermore, the room-temperature molten salt of the present invention can be obtained by mixing two or more organic salts selected from the group consisting of the organic salts represented by formula (VI). Of the two or more starting organic salts, preferably at least one, and more preferably all are solids at room temperature.

Moreover, the room-temperature molten salt of the present invention can be obtained by mixing at least one organic salt that is selected from the group consisting of the organic salts represented by formula (V) and is a solid at room temperature, and at least one organic salt that is selected from the group consisting of the organic salts represented by formula (VI) and is a solid at room temperature.

The substituents in formulae (I) to (IV) are as defined above, and examples thereof are as follows. (i) R^{1a} to R^{5a}, R^{7a}, R^{9a} and R^{10a} Examples of halogen atoms include fluorine, chlorine, bromine and iodine atoms.

Examples of alkyl groups include straight- or branched-chain C₁₋₁₀ alkyl groups, and preferable are straight- or branched-chain C₁₋₆ alkyl groups such as methyl, ethyl, n-propyl, isopropyl, n-butyl, isobutyl, sec-butyl, tert-butyl, n-pentyl, n-hexyl, neopentyl, isohexyl, etc.

Examples of cycloalkyl groups include C₃₋₁₀ cycloalkyl groups, and preferable are C₃₋₆ cycloalkyl groups such as cyclopropyl, cyclobutyl, cyclopentyl, cyclohexyl, etc.

Examples of heterocyclic groups include three- to six-membered aliphatic and aromatic mono-heterocyclic groups with at least one hetero atom selected from the group consisting of nitrogen atoms, oxygen atoms and sulfur atoms. Specific examples include aziridinyl, pyrrolidinyl, piperidyl, piperazinyl, morpholinyl, tetrahydropyranyl, pyridyl, furyl, thienyl, etc. Substituent(s) may be bonded to such heterocyclic groups. Examples of substituents include fluorine, chlorine and like halogen atoms; methyl, ethyl and like alkyl groups; trifluoromethyl and like haloalkyl groups; methoxy, ethoxy and like alkoxy groups; phenyl and like aryl groups; etc.

Examples of haloalkyl groups include alkyl groups in which at least one hydrogen atom is substituted by halogen, and preferable are straight- or branched-chain C₁₋₁₀ alkyl groups in which at least one hydrogen atom is substituted by fluorine. Specific examples include trifluoromethyl, trifluoroethyl, trichloroethyl, tetrafluoroethyl, perfluoroethyl, perfluoropropyl, perfluoroisopropyl, perfluorobutyl, perfluorohexyl, perfluorooctyl, perfluorodecyl, 2-(perfluorooctyl)ethyl, 1H,1H,3H-tetrafluoropropyl, 1H,1H,5H-octafluoropentyl, etc. Among these, especially preferable are C₁₋₆ straight- or branched-chain alkyl groups in which at least one hydrogen atom is substituted by fluorine, such as trifluoromethyl, trifluoroethyl, trichloroethyl, tetrafluoroethyl, perfluoroethyl, perfluoropropyl, perfluoroisopropyl, perfluorobutyl, perfluorohexyl, etc.

Examples of aralkyl groups include C₇₋₁₀ aralkyl groups. Specific examples include 2-phenylethyl, benzyl, 1-phenylethyl, 3-phenylpropyl, 4-phenylbutyl, etc.

Examples of aryl groups include phenyl groups, naphthyl groups, etc. Substituent(s) may be bonded to such aryl groups. Examples of substituents include fluorine, chlorine and like halogen atoms; methyl, ethyl and like alkyl groups; trifluoromethyl and like haloalkyl groups; methoxy, ethoxy and like alkoxy groups; phenyl and like aryl groups; etc.

Examples of alkoxy groups include straight- or branched-chain C₁₋₁₀ alkoxy groups, and preferable are straight- or branched-chain C₁₋₆ alkoxy groups such as methoxy, ethoxy, n-propoxy, isopropoxy, n-butoxy, isobutoxy, sec-butoxy, tert-butoxy, pentyloxy, hexyloxy, etc.

Examples of aryloxy groups include phenoxy, naphthyloxy and like groups.

Examples of aralkyloxy groups include C₇₋₁₀ aralkyloxy groups, and specific examples include 2-phenylethyloxy, benzyloxy, 1-phenylethyloxy, 3-phenylpropyloxy, 4-phenylbutyloxy, etc.

(ii) R^{8a}

Examples of alkyl, cycloalkyl, heterocyclic, haloalkyl, aralkyl and aryl groups are as described above.

(iii) R^{6a}, R^{11a}, R¹², R¹³, R¹⁴, R¹⁵, R¹⁶, R¹⁷, R¹⁸ and R¹⁹

Examples of alkyl, cycloalkyl, heterocyclic, haloalkyl, aralkyl and aryl groups are as described above.

When R^{6a} and R^{11a} are haloalkyl groups, C₁₋₁₀ alkyl groups in which at least one hydrogen atom is substituted by fluorine are preferable. Examples of such haloalkyl groups include straight- or branched-chain C₁₋₁₀ perfluoroalkyl groups, straight- or branched-chain C₁₋₁₀ polyfluoroalkyl groups, etc. Specific examples of straight- or branched-chain C₁₋₁₀ perfluoroalkyl groups include perfluoroethyl, perfluoropropyl, perfluorobutyl, perfluoropentyl, perfluorohexyl, perfluoroheptyl, perfluorooctyl, perfluorononyl, perfluorodecyl, etc. Examples of straight- or branched-chain C₁₋₁₀ polyfluoroalkyl groups include the above-mentioned alkyl groups in which at least one hydrogen atom is substituted by fluorine. Specific examples of such polyfluoroalkyl groups include CF₃CH₂, CF₃CF₂CH₂, CF₃CF₂CF₂CH₂, CF₃CF₂(CH₂)₆, HCF₂CF₂CH₂, HCF₂CF₂CF₂CF₂CH₂, H(CF₂)₆CH₂, CF₃CHFCF₂CH₂, (CF₃)₂CH, (CF₃)₂CHCH₂, (CF₃)₂C(CH₃)CH₂, etc.

Particularly preferable examples of R⁶ and R¹¹ include groups represented by the formula —CH₂R¹² wherein R¹² is a straight- or branched-chain C₁₋₉ alkyl group in which at least one hydrogen atom is substituted by fluorine. Preferable examples of R¹² include straight- or branched-chain C₁₋₆ alkyl groups in which at least one hydrogen atom is substituted by fluorine, such as fluoromethyl, difluoromethyl, trifluoromethyl, perfluoroethyl, perfluoropropyl, CF₃CF₂(CH₂)₅, HCF₂CF₂, H(CF₂)₄, H(CF₂)₆, (CF₃)₂CH, CF₃CHFCF₂, etc.

When two groups selected from R¹², R¹³, R¹⁴ and R¹⁵ are linked at their ends to form, together with the adjacent nitrogen atom, a nitrogen-containing aliphatic heterocycle, examples of the nitrogen-containing aliphatic heterocycles include three- to ten-membered nitrogen-containing aliphatic heterocycles, and specific examples include aziridine, pyrrolidine, piperidine, morpholine, perhydro-2H-azepine, etc.

When two groups selected from R¹⁶, R¹⁷, R¹⁸ and R¹⁹ are linked at their ends to form, together with the adjacent phosphorus atom, a phosphorus-containing aliphatic heterocycle, the phosphorus-containing aliphatic heterocycle may be, for example, a three- to ten-membered phosphorus-containing aliphatic heterocycle. Specific examples include phosphirane, phosphetane, phosphol, etc.

(vi) X₁⁻, X₂⁻, X₃⁻ and X₄⁻

X₁⁻, X₂⁻, X₃⁻ and X₄⁻ each represent an anionic moiety of the individual starting organic salts. The anionic moiety is a conjugate base of a Brönsted acid. Examples of such Brönsted acids include Brönsted acids with strong acidity, such as sulfuric acid; monomethyl sulfate, monoethyl sulfate and like sulfuric acid monoesters; methansulfonic acid, ethanesulfonic acid, chlorosulfonic acid, fluorosulfonic acid, benzenesulfonic acid, toluenesulfonic acid, nitrobenzenesulfonic acid, trichloromethanesulfonic acid, acids represented by the formula Rf″SO₃H wherein Rf″ is a polyfluoroalkyl group, and like sulfonic acids; sulfonimides represented by the formula (RfSO₂)₂NH or (RfSO₂)(Rf′SO₂)NH wherein Rf and Rf′ are different and each represents a polyfluoroalkyl group; formic acid, acetic acid, butyric acid, valeric acid, trifluoroacetic acid, perfluorobutyric acid, perfluorooctanoic acid, 3H-octafluorobutyric acid, trichloroacetic acid and like carboxylic acids; HB(OCOCF₃)₄, HB(OCOC₂F₅)₄, HBPh₄, HB(C₆F₅)₄, HB(p-CF₃C₆H₄)₄, HB[3,5-(CF₃)₂C₆H₃], HC(SO₂CF₃)₃, HC(SO₂C₂F₅)₃ and like organic acids; HBF₄, HPF₆, HSbF₆, HAsF₆, HBCl₄, HBCl₃F, HSbCl₆, HSbCl₅F, HClO₄, HNO₃, HAlCl₄, HAl₂Cl₇ and like inorganic acids; etc.

The polyfluoroalkyl groups represented by Rf, Rf′ and Rf″ may be the same or different and may be independently a straight- or branched-chain C₁₋₆ perfluoroalkyl group or a straight- or branched-chain C₁₋₆ alkyl group in which at least one hydrogen atom is substituted by fluorine. Specific examples include trifluoromethyl, pentafluoroethyl, trifluoroethyl, perfluoropropyl, perfluorobutyl, etc.

Examples of acids represented by the formula Rf″SO₃H include C₄F₉SO₃H, CF₃SO₃H, CF₃CF₂SO₃H, CF₃CH₂SO₃H, HCF₂CF₂CH₂SO₃H, C₆Fl₃SO₃H, HCF₂CF₂CF₂CF₂SO₃H, etc.

Examples of sulfonimides include (CF₃SO₂)₂NH, (C₂F₅SO₂)₂NH, (C₄F₉SO₂)₂NH, (CF₃SO₂)(C₄F₉SO₂)NH, (C₂F₅SO₂)(C₄F₉SO₂)NH, (HCF₂CF₂SO₂)₂NH, (CF₃CH₂SO₂)(C₄F₉SO₂)NH, etc.

As mentioned above, the organic salts for use as the starting materials of the room-temperature molten salt of the present invention all have different anionic moieties (conjugate bases of Brönsted acids).

Preferable organic salts for use as starting materials include organic salts of formulae (V) and (VI) wherein R¹ to R⁵, R⁷, R⁹ and R¹⁰ are the same or different and each represents a hydrogen atom, a halogen atom, an alkyl group or a haloalkyl group; R⁸ is an alkyl group; R⁶ and R¹¹ are the same or different and each represents a group represented by the formula —CH₂R¹² wherein R¹² is a straight- or branched-chain C₁₋₉ alkyl group in which at least one hydrogen atom is substituted by fluorine.

With respect to the substituents of the organic salts of formulae (V) and (VI), examples of R¹ to R⁵, R⁷, R⁸ and R⁹ and R¹⁰ are the same as those of R^{1a} to R^{5a}, R^{7a}, R^{8a}, R^{9a} and R^{10a}, respectively.

In R⁶ and R¹¹ represented by the formula —CH₂R¹² wherein R¹² is a straight- or branched-chain C₁₋₉ alkyl group in which at least one hydrogen atom is substituted by fluorine, preferable examples of R¹² include fluoromethyl, difluoromethyl, trifluoromethyl, perfluoroethyl, perfluoropropyl, CF₃CF₂(CH₂)₅, HCF₂CF₂, H(CF₂)₄, H(CF₂)₆, (CF₃)₂CH, CF₃CHFCF₂ and like straight- or branched-chain C₁₋₆ alkyl groups in which at least one hydrogen atom is substituted by fluorine.

Preferably, the room-temperature molten salt of the present invention is obtained by mixing two or more organic salts that are selected from the group consisting of the organic salts represented by formulae (V) and (VI) and are solid at room temperature. It is particularly preferable to obtain the room-temperature molten salt by mixing two organic salts that are selected from the group consisting of the organic salts represented by formulae (V) and (VI) and are solid at room temperature. Preferable examples of the two organic salts include the combination of an organic salt with an anionic moiety represented by the formula
(RfSO₂)₂N⁻ or (RfSO₂)(Rf′SO₂)N⁻
wherein Rf and Rf′ are different and each represents a polyfluoroalkyl group, and an organic salt with an anionic moiety represented by the formula
Rf″ SO₃⁻
wherein Rf″ is a polyfluoroalkyl group.

Of the room-temperature molten salts according to the present invention, preferable are those obtained by mixing organic salts selected from the following <organic salts>, and those obtained by mixing two or more organic salts selected from the following <organic salts>. In particular, room-temperature molten salts obtained by mixing two or three organic salts selected from the following <organic salts> are preferable. The following <organic salts> are preferably solids at room temperature. “Tf⁻” in the organic salts means a trifluoromethanesulfonyl group (CF₃SO₂—). 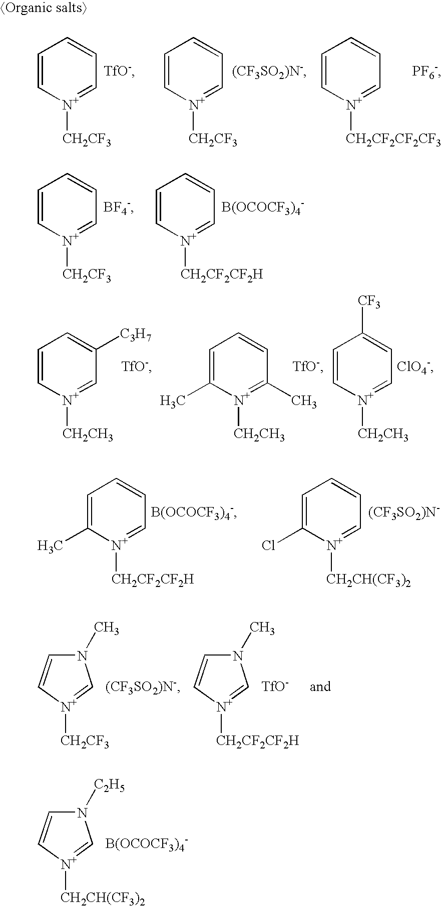
Process for Producing the Room-temperature Molten Salt of the Present Invention

The starting organic salts of formulae (I) to (VI) for the room-temperature molten salt of the present invention can be synthesized by, for example, the processes described in Inorg. Chem. (1996) 35, 1168, and Bull. Chem. Soc. Jpn., (1991) 64, 2008.

The room-temperature molten salt of the present invention is produced by mixing two or more organic salts with different anionic moieties and different organic cationic moieties. In the production process, the mixing ratio of the two or more organic salts selected from the group consisting of the organic salts represented by formulae (I) to (VI) is not limited, and is suitably selected so that the organic salts can be mixed to form a uniform liquid. A suitable mixing ratio is, for example, 100 parts by weight of one of the organic salts and about 1 to about 1000 parts by weight, preferably about 10 to about 500 parts by weight, and more preferably about 30 to about 300 parts by weight of each of the other organic salts. In order to obtain a room-temperature molten salt with a low solidifying point, it is preferable to mix approximately equal weights of the two or more organic salts.

The solidifying point of the room-temperature molten salt of the present invention varies with the types and mixing ratio of the starting organic salts, and is usually about 10° C. lower, preferably about 20° C. lower, more preferably about 50° C. lower, and particularly preferably 80° C. lower, than the organic salt with the lowest solidifying point of all the starting organic salts used. For example, when the room-temperature molten salt is produced by mixing approximately equal weights of two organic salts that are selected from the group consisting of the organic salts represented by formulae (V) and (VI) and are solids at room temperature, the room-temperature molten salt may have a solidifying point about 50° C. to about 100° C. lower than that of the organic salt with the lowest solidifying point. In particular, when organic salts selected from the above <organic salts> are mixed, the resulting room-temperature molten salt of the present invention has a greatly lowered solidifying point than any of the individual starting organic salts.

The method of mixing the organic salts is not limited, and may be a known method, for example, mixing in a mortar, mixing with a stirrer, or mixing with heating. When using the room-temperature molten salt of the present invention as a nonaqueous battery electrolyte or the like, it is preferable to mix the organic salts in a dry atmosphere to prevent moisture from being admixed.

In particular, when the starting organic salts are solids around room temperature, purification is very easy since organic and inorganic impurities can be removed by a simple process, such as washing, recrystallization or the like. Thus, use of starting organic salts that are solid at room temperature makes it possible to obtain the room-temperature molten salt of the present invention in high purity. Accordingly, to obtain the room-temperature molten salt of the present invention in high purity, it is preferable that at least one of the starting organic salts is a solid at room temperature, and more preferably all of the starting organic salts are solids at room temperature.

The room-temperature molten salt of the present invention thus obtained has almost no vapor pressure, has high heat resistance, is liquid over a wide temperature range because of its low solidifying point, and has a high ionic conductivity. Moreover, especially when containing fluorine in the molecule, the room-temperature molten salt has the features of high flame retardancy and low viscosity.

Since the room-temperature molten salt of the present invention is a mixture, it sometimes does not exhibit a clear solidifying point. Therefore, in this specification and the appended claims, “solidifying point” of the room-temperature molten salt of the present invention is intended to mean the value measured by the method described in Experiment 1. Specifically, the room-temperature molten salt of the present invention is placed in an inert gas (e.g., argon) atmosphere in an airtight container and cooled at a rate of 2 to 3° C./min, and the temperature at which the beginning of precipitation of the room-temperature molten salt as a solid is observed with the naked eye is determined as the “solidifying point”.

The “solidifying point” of the room-temperature molten salt as defined above can be measured with good reproducibility by the method of Experiment 1, but supercooling may occur before the precipitation of solids. Therefore, in addition to the above solidifying point measurement, the precipitation temperature (glass transition temperature) of an amorphous solid of the room-temperature molten salt was measured using a differential scanning calorimeter (DSC).

These measurements reveal that some of the room-temperature molten salts according to the present invention have the feature of not having a melting point and not undergoing a phase change (primary phase change) in a temperature range from room temperature to extremely low temperatures. For example, this feature is noticeable in preferable room-temperature molten salts of the invention obtained by mixing two or more organic salts that are selected from the group consisting of the organic salts represented by formulae (V) and (VI) and are solids at room temperature.

Specifically, as shown in Experiment 2, differential scanning calorimeter (DSC) measurements were performed on room-temperature molten salts according to the present invention (Examples 1, 3 and 4) and 1-ethyl-3-methylimidazolium trifluoromethanesulfonate, i.e., a known room temperature-molten salt (Comparative Example 5). The room-temperature molten salt of Comparative Example 5 was observed to have a melting point (Tm) around −13° C., while in respect of the room-temperature molten salts of Examples 1, 3 and 4, no melting point (Tm) was observed and only a glass transition temperature (Tg) was observed around −50° C. These results demonstrate that the room-temperature molten salts of the present invention obtained in Examples 1, 3 and 4 undergo no phase change until they reach the glass transition temperature around −50° C. Thus, salts that initially have a melting point can be easily converted, by being mixed, to a room-temperature molten salt that has no melting point and does not undergo a phase change until it reaches the glass transition temperature.

Use of the Room-temperature Molten Salt of the Present Invention

Because of the above features, the room-temperature molten salt of the present invention can be used, singly or in combination with a solvent heretofore used in electrolytic solutions, as an electrolyte or electrolytic solution for lithium ion (primary or secondary) batteries. Examples of solvents heretofore used in electrolytic solutions include known nonaqueous organic solvents such as propylene carbonate, ethylene carbonate, diethyl carbonate, dimethyl carbonate, methyl ethyl carbonate, dimethoxyethane, γ-butyrolactone, methyl acetate, methyl formate, etc. The room-temperature molten salt of the present invention can be added as an electrolyte or a part of an electrolytic solution to these solvents to form an electrolytic solution. The electrolytic solution comprising the room-temperature molten salt of the present invention may further contain LiPF₆, LiPF₄(CF₃)₂, LiPF₄(C₂F₅)₂, LiPF₄(C₃F₇)₂, LiAsF₆, LiBF₄, LiClO₄, LiCF₃SO₃, LiC₄F₉SO₃, LiN(CF₃SO₂)₂, LiN(C₂F₅SO₂)₂, LiN(C₄F₉SO₂)₂, LiN(CF₃SO₂)(C₄F₉SO₂), LiC(CF₃SO₂)₃ and/or like lithium salts as an electrolyte. The concentration of the lithium salt electrolyte(s) is not limited, and a concentration of 0.5 mol/l to 1.5 mol/l is usually practical. Of course, it is preferable that the electrolytic solution have a water content not greater than 10 ppm.

The above electrolyte and the room-temperature molten salt of the present invention can be used as an electrolyte for nonaqueous solutions with lithium ion conductivity, or for a gel electrolyte comprising the electrolyte immobilized in a polymer matrix, as described in, for example, J. Electrochem. Soc., (2000) 147, 34.

Especially when the room-temperature molten salt of the present invention is produced by mixing organic salts that are solids at room temperature, the starting organic salts can be purified by recrystallization or like processes, and as a result of the purification, the obtained room-temperature molten salt is free of inorganic salt impurities. Therefore, such a room-temperature molten salt is especially preferable for use as lithium ion (primary or secondary) battery electrolytes or electrolytic solutions which are required to have high purity.

Moreover, as mentioned above, the room-temperature molten salt of the present invention is liquid over a wide temperature range, and thus the lithium ion battery of the present invention, which comprises the room-temperature molten salt as an electrolyte or a part of an electrolytic solution, has the feature of exhibiting stable battery characteristics in environments with a wide temperature range (e.g., in cold districts).

In producing a lithium ion (primary or secondary) battery, known positive and negative electrodes, separators and the like can be used as such.

The battery may have the shape of, for example, a cylinder, square, button, film or the like.

Examples of negative electrode materials include lithium metals and alloys thereof, carbon or polymer materials capable of being doped and dedoped with lithium, lithium-intercalated compounds such as metal oxides, etc.

Examples of positive electrode materials include complex oxides of lithium and a transition metal, such as LiCoO₂, LiNiO₂, LiMn₂O₄ and LiMnO₂, polymer materials, etc.

Usable separators include, for example, porous membranes of polymer materials such as polyethylene and polypropylene, polymer materials capable of immobilizing the electrolytic solution of the present invention (so-called gel electrolyte), etc.

Examples of collector materials include copper, aluminum, stainless steel, titanium, nickel, tungsten steel, carbon materials, etc. The collector may be in the form of, for example, a foil, net, nonwoven fabric, punched metal or the like.

The room-temperature molten salt of the present invention can be used as a solvent in various organic synthesis reactions. The room-temperature molten salt has low solubility in water, and in particular, when the anionic moiety of the organic salts forming the room-temperature molten salt is Rf″SO₃⁻, (RfSO₂)₂N⁻ or (RfSO₂)(Rf′SO₂)N⁻ wherein Rf, Rf′ and Rf″ are as defined above, Ph₄B⁻, (C₆H₅)₄B⁻, (p-CF₃C₆H₄)₄B⁻, [3,5-(CF₃)₂C₆H₃]₄B⁻ or the like, the room-temperature molten salt has extremely low solubility in water. Such a room-temperature molten salt makes it possible to construct a two-phase reaction system consisting of water and the room-temperature molten salt. Moreover, since the room-temperature molten salt is sparingly soluble in low-polarity organic solvents (e.g., toluene, ethyl acetate, diethyl ether, etc.), it is also possible to construct a three-phase reaction system consisting of an organic solvent, water and the room-temperature molten salt. Further, the room-temperature molten salt has high heat resistance and therefore enables the selection of reaction conditions from a wide temperature range. Furthermore, the room-temperature molten salt, after being used as a reaction solvent, can be used as an extraction solvent for separation and purification as described below.

The room-temperature molten salt of the present invention can also be used as an extraction solvent for separation and purification in organic synthesis reactions. For example, in a post-treatment of a reaction mixture containing a catalyst (e.g., a metal catalyst), when the reaction solvent is distilled off and then an ether and the room-temperature molten salt of the present invention are added to the residue, a two-phase system is formed in which the reaction product and catalyst are held in the ethereal phase and room-temperature molten salt phase, respectively. Therefore, the reaction product can be easily separated from the catalyst and purified. Moreover, in some types of reactions, the catalyst held in the room-temperature molten salt retains its activity and can be recycled. Thus, the room-temperature molten salt of the present invention is extremely useful as an environmentally friendly solvent (see Chemistry, vol. 56, No.5 (2001)).

Furthermore, the room-temperature molten salt of the present invention has high heat resistance, is liquid over a wide temperature range and has high ion conductivity as mentioned above, and therefore can also be used as an electrolytic solution for plating.

The room-temperature molten salt of the present invention does not undergo a phase change until it reaches an extremely low temperature, and has excellent low temperature properties. Because of these characteristics, the room-temperature molten salt is usable, besides in the above applications, as an electrolyte and/or electrolytic solution for fuel cells (in particular polymer electrolyte fuel cells), dye-sensitized solar cells, biological batteries or capacitors (in particular electric double layer capacitors); an electro-rheological fluid; a heat storage medium; a catalyst; etc.

### BRIEF EXPLANATION OF THE DRAWINGS

FIGS. 1 to 4 are graphs indicating the results of the differential scanning calorimeter (DSC) measurements of the room-temperature molten salts of Examples 1, 3 and 4 and Comparative Example 5, respectively.

### BEST MODE FOR CARRYING OUT THE INVENTION

The following Examples illustrate the present invention in further detail, but are not intended to limit the scope thereof.

A. Synthesis of Starting Organic Salts

### REFERENCE EXAMPLE 1

Synthesis of 1-(2,2,2-trifluoroethyl)-3-methylpyridinium trifluoromethanesulfonate

3-methylpyridine (5 mmol, 487 FL) and 2,2,2-trifluoroethyl trifluoromethanesulfonate (5 mmol, 1.16 g) in 1,1,1-trichloroethane (2 mL) were heated and refluxed for 1.5 hours. The layered reaction mixture was separated, and the reaction product was washed with 1,1,1-trichloroethane (2 mL) and vacuum-dried to thereby obtain a brown solid (865 mg, yield: 53.2%). Melting point: 67.7 to 68.9° C.

¹H-NMR (CD₃CN): δ2.55 (s, 3H), 5.29 (q, J=8.2, 2H), 8.04 (dd, J=6.2, 8.0, 1H), 8.50 (d, J=8.0, 1H), 8.62 (d, J=6.2, 1H), 8.64 (s, 1H)

¹⁹F-NMR (CD₃CN): δ−78.08 (s, 3F), −70.46 (t, J=8.2, 3F)

### REFERENCE EXAMPLE 2

Synthesis of 1-(2,2,2-trifluoroethyl)-4-methylpyridinium trifluoromethanesulfonate

The procedure of Reference Example 1 was followed to synthesize the title compound from the corresponding starting compounds. Yield: 99%. Melting point: 100.0 to 101.0° C.

¹H-NMR (CD₃CN): δ2.68 (s, 3H), 5.29 (q, J=8.5, 2H), 7.96 (d, J=6.5, 2H), 8.62 (d, J=6.5, 2H)

¹⁹F-NMR (CD₃CN): δ−78.11 (s, 3F), −70.80 (t, J=8.5, 3F)

### REFERENCE EXAMPLE 3

Synthesis of 1-(2,2,3,3-tetrafluoropropyl)-2-methylpyridinium trifluoromethanesulfonate

The procedure of Reference Example 1 was followed to synthesize the title compound from the corresponding starting compounds. Yield: 99%. Melting point: 79.0 to 80.5° C.

¹H-NMR (acetone-d₆): δ3.09 (s, 3H), 5.71 (t, J=15.6, 2H), 6.76 (tt, J=52.2, 4.7, 1H), 8.18-9.19 (m, 4H)

¹⁹F-NMR (acetone-d₆): δ−137.71 (dt, J=4.3, 52.2, 2F), −120.80-−120.50 (m, 2F), −78.25 (s, 3F).

### REFERENCE EXAMPLE 4

Synthesis of 1-methyl-3-(2,2,2-trifluoroethyl)imidazolium trifluoromethanesulfonate

The procedure of Reference Example 1 was followed to synthesize the title compound from the corresponding starting compounds. Yield: 94%. Melting point: 51.0 to 51.9° C.

¹H-NMR (acetone-d₆): δ4.15 (s, 3H), 5.42 (q, J=8.6, 2H), 7.85-7.95 (m, 2H), 9.34 (s, 1H)

19F-NMR (acetone-d₆): δ−79.18 (s, 3F), −71.53 (t, J=8.6, 3F)

### REFERENCE EXAMPLE 5

Synthesis of 1-(2,2,2-trifluoroethyl)pyridinium bis[(trifluoromethyl)sulfonyl]amide

1-(2,2,2-trifluoroethyl)pyridinium trifluoromethanesulfonate (4.8 mmol, 1.50 g), synthesized from the corresponding starting compounds by following the procedure of Reference Example 1, and LiN(SO₂CF₃)₂ (4.8 mmol, 1.38 g) were heated in water (7.2 mL) at 70° C. for 4 hours. The layered reaction mixture was separated, and the reaction product was washed with 1,1,1-trichloroethane (2 mL) and water (2 mL) and vacuum-dried to thereby obtain a white solid (1.87 g, yield: 88%). Melting point: 38.3 to 38.8° C.

¹H-NMR (acetone-d₆): δ5.93 (q, J=8.2, 2H), 8.43-9.50 (m, 5H)

¹⁹F-NMR (acetone-d₆): δ−78.97 (s, 6F), −70.91 (t, J=8.2, 3F)

### REFERENCE EXAMPLE 6

Synthesis of 1-(2,2,2-trifluoroethyl)-4-methylpyridinium bis[(trifluoromethyl)sulfonyl]amide

The procedure of Reference Example 5 was followed to synthesize the title compound from the corresponding starting compounds. Yield: 71.9%. Melting point: 60.3 to 61.1° C.

¹H-NMR (CD₃CN): δ2.71 (s, 3H), 5.27 (q, J=8.2, 2H), 7.98 (d, J=6.5, 2H), 8.59 (d, J=6.5, 2H)

¹⁹F-NMR (CD₃CN): δ−78.95 (s, 6F), −70.79 (t, J=8.2, 3F)

B. Production of the Room-temperature Molten Salt of the Present Invention

### EXAMPLE 1

1-(2,2,2-trifluoroethyl)-3-methylpyridinium trifluoromethanesulfonate (30 mg) and 1-(2,2,2-trifluoroethyl)pyridinium bis[(trifluoromethyl)sulfonyl]amide (30 mg) were fully mixed in a mortar in a dry atmosphere, to thereby obtain a product that was a colorless transparent liquid at room temperature.

### EXAMPLE 2

1-(2,2,2-trifluoroethyl)-4-methylpyridinium trifluoromethanesulfonate (30 mg) and 1-(2,2,2-trifluoroethyl)pyridinium bis[(trifluoromethyl)sulfonyl]amide (30 mg) were mixed in the same manner as in Example 1, to thereby obtain a product that was a light yellow transparent liquid at room temperature.

### EXAMPLE 3

1-(2,2,3,3-tetrafluoropropyl)-2-methylpyridinium trifluoromethanesulfonate (30 mg) and 1-(2,2,2-trifluoroethyl)pyridinium bis[(trifluoromethyl)sulfonyl]amide (30 mg) were mixed in the same manner as in Example 1, to thereby obtain a product that was a colorless transparent liquid at room temperature.

### EXAMPLE 4

1-methyl-3-(2,2,2-trifluoroethyl)imidazolium trifluoromethanesulfonate (30 mg) and 1-(2,2,2-trifluoroethyl)pyridinium bis[(trifluoromethyl)sulfonyl]amide (30 mg) were mixed in the same manner as in Example 1, to thereby obtain a product that was a light yellow transparent liquid at room temperature.

### EXAMPLE 5

1-(2,2,2-trifluoroethyl)-3-methylpyridinium trifluoromethanesulfonate (30 mg) and 1-(2,2,2-trifluoroethyl)-4-methylpyridinium bis[(trifluoromethyl) sulfonyl]amide (30 mg) were fully mixed in the same manner as in Example 1, to thereby obtain a product that was a light yellow transparent liquid at room temperature.

### EXAMPLE 6

1-(2,2,3,3-tetrafluoropropyl)-2-methylpyridinium trifluoromethanesulfonate (30 mg) and 1-(2,2,2-trifluoroethyl)-4-methylpyridinium bis[(trifluoromethyl) sulfonyl]amide (30 mg) were fully mixed in the same manner as in Example 1, to thereby obtain a product that was a colorless transparent liquid at room temperature.

### EXAMPLE 7

1-(2,2,2-trifluoroethyl)-3-methylpyridinium trifluoromethanesulfonate (30 mg), 1-(2,2,2-trifluoroethyl)pyridinium bis[(trifluoromethyl)sulfonyl]amide (30 mg) and 1-(2,2,2-trifluoroethyl)-4-methylpyridinium bis[(trifluoromethyl)sulfonyl]amide (30 mg) were fully mixed in a mortar in a dry atmosphere, to thereby obtain a colorless transparent product that was liquid at room temperature.

### EXAMPLE 8

1-methyl-2-ethylpyridinium trifluoromethanesulfonate (30 mg) and 1-(2,2,2-trifluoroethyl)pyridinium bis[(trifluoromethyl)sulfonyl]amide (30 mg) were mixed in the same manner as in Example 1, to thereby obtain a colorless transparent product that was liquid at room temperature.

The solidifying point of 1-methyl-2-ethylpyridinium trifluoromethanesulfonate measured by the method described in Experiment 1 was −39° C.

### EXAMPLE 9

1-methyl-2-ethylpyridinium trifluoromethanesulfonate (30 mg) and 1-methyl-3-(2,2,2-trifluoroethyl)imidazolium bis[(trifluoromethyl)sulfonyl]amide (30 mg) were mixed in the same manner as in Example 1, to thereby obtain a colorless transparent product that was liquid at room temperature.

The solidifying point of 1-methyl-3-(2,2,2-trifluoroethyl)imidazolium bis[(trifluoromethyl)sulfonyl]amide measured by the method described in Experiment 1 was −66° C.

Experiment 1 (Measurement of Solidifying Point)

The solidifying points of the room-temperature molten salts obtained in Examples 1 to 7 were measured by the following method: Each room-temperature molten salt was placed in an argon atmosphere in an airtight container and cooled at a rate of 2° C. to 3° C./min. The temperature at which the room-temperature molten salt began to precipitate as a solid was determined as the solidifying point. Table 1 shows the measurement results.

TABLE 1
Solidifying point of room-temperature molten salt

| | Solidifying |
|---|---|
| Example | point (° C.) |
| 1 | −87.0 |
| 2 | −60.0 |
| 3 | −78.7 |
| 4 | −85.0 |
| 5 | −72.7 |
| 6 | −72.8 |
| 7 | <−90.0 |
| 8 | <−90.0 |
| 9 | <−90.0 |

Table 1 reveals that all the room-temperature molten salts had a very low solidifying point.

### COMPARATIVE EXAMPLE 1

1-(2,2,2-trifluoroethyl)-3-methylpyridinium trifluoromethanesulfonate (30 mg) and 1-(2,2,2-trifluoroethyl)-4-methylpyridinium trifluoromethanesulfonate (30 mg) were mixed in the same manner as in Example 1, but the resulting mixture remained solid at room temperature.

### COMPARATIVE EXAMPLE 2

1-(2,2,2-trifluoroethyl)-2-methylpyridinium trifluoromethanesulfonate (30 mg) and l-(2,2,2-trifluoroethyl)-4-methylpyridinium trifluoromethanesulfonate (30 mg) were mixed in the same manner as in Example 1, but the resulting mixture remained solid at room temperature.

### COMPARATIVE EXAMPLE 3

1-(2,2,2-trifluoroethyl)-4-methylpyridinium bis[(trifluoromethyl)sulfonyl]amide (30 mg) and 1-(2,2,2-trifluoroethyl)pyridinium bis[(trifluoromethyl)sulfonyl]amide (30 mg) were mixed in the same manner as in Example 1, but the resulting mixture remained solid at room temperature.

### COMPARATIVE EXAMPLE 4

1-(2,2,2-trifluoroethyl)-4-methylpyridinium trifluoromethanesulfonate (30 mg) and 1-(2,2,2-trifluoroethyl)-4-methylpyridinium bis[(trifluoromethyl) sulfonyl]amide (30 mg) were mixed in the same manner as in Example 1, but the resulting mixture remained solid at room temperature.

As is apparent from the above, when two or more different organic salts with the same anionic or cationic moiety are mixed, the solidifying point lowers only slightly as compared with in the room-temperature molten salt of the present invention, and the resulting mixture is not liquid at room temperature. This matter is specifically demonstrated by Comparative Examples 1, 2 and 3 in which two types of organic salts with the same anionic moiety were mixed, and in Comparative Example 4 in which two types of organic salts with the same cationic moiety were mixed.

In contrast, when organic salts that were different not only in cationic moiety but also in anionic moiety were mixed, the resulting mixture was liquid and had a remarkably lowered solidifying point. Comparison between Example 2 and Comparative Example 3 illustrates this point.

Experiment 2 (Differential Scanning Calorimeter (DSC) Measurement)

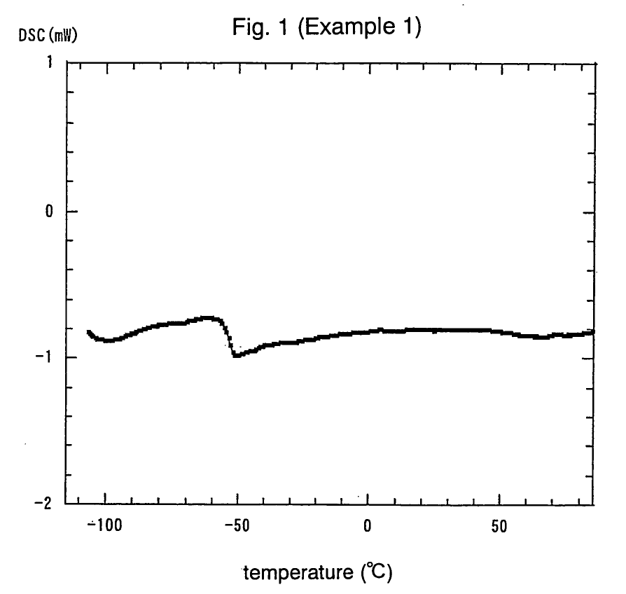

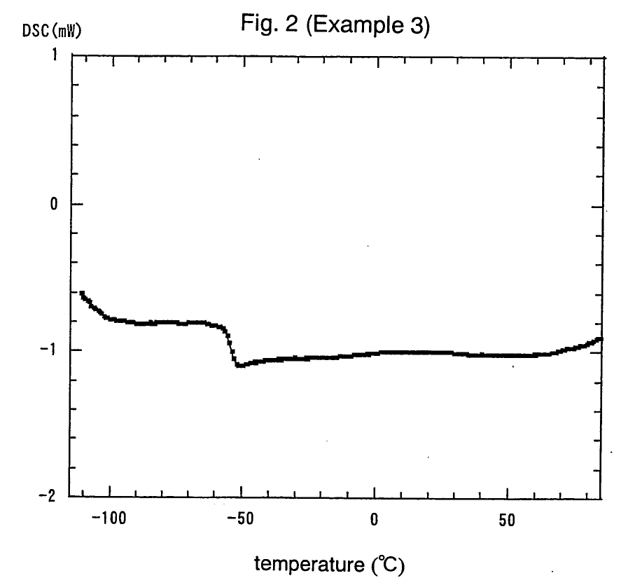

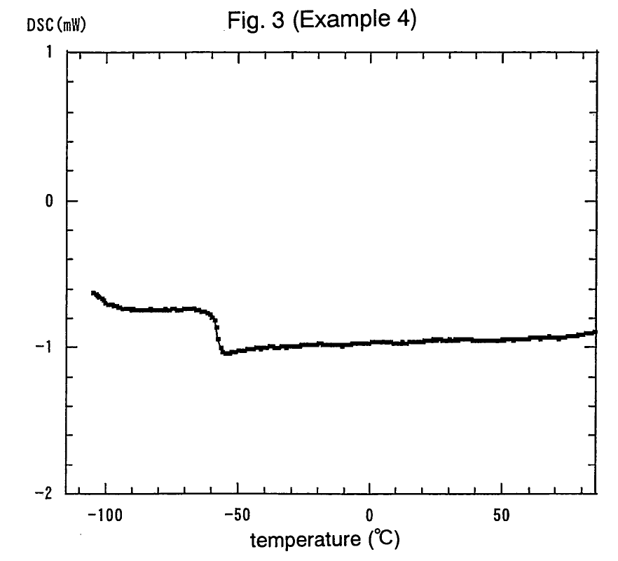

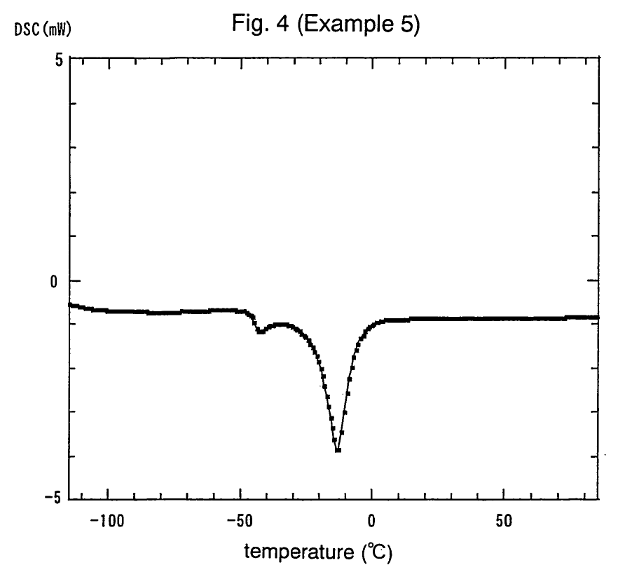

Differential scanning calorimeter (DSC) measurements were carried out on the room-temperature molten salts of Examples 1, 3 and 4 and on 1-ethyl-3-methylimidazolium trifluoromethanesulfonate, which is a room-temperature molten salt obtained from Aldrich (Comparative Example 5). A DSC-50 of Shimadzu Corp. was used for the measurements. Five milligrams of sample was weighed out, sealed in an aluminium cell, and placed in the DSC chamber together with a reference (an empty aluminium cell). While being purged with nitrogen at a rate of 20 ml/min, the chamber was cooled from room temperature to −120° C. at a rate of 1° C. to 5° C./min using liquid nitrogen. After being maintained at −120° C. for 30 minutes, the chamber temperature was raised to 100° C. at a rate of 10° C./min, and the data obtained during the temperature rise was collected. FIGS. 1 to 4 shows the measurement results.

The figures reveal that the melting point (Tm) was observed around −13° C. in Comparative Example 5, whereas in Examples 1, 3 and 4, no melting point (Tm) was observed and only the glass transition temperature (Tg) was observed around −60° C.

### INDUSTRIAL APPLICABILITY

According to the present invention, at least two types of organic salts that are different from each other both in anionic moiety and cationic moiety are mixed, with the result that the solidifying point is remarkably lowered and a liquid mixed organic salt (room-temperature molten salt) with a lower solidifying point is obtained.

Preferably, at least one, and more preferably all, of the organic salts to be mixed are solids at room temperature. By mixing organic salts that are solids at room temperature, the solidifying point is lowered, thereby giving a mixed organic salt that is liquid at room temperature (room-temperature molten salt). In this case, the starting organic salts are all solids at room temperature, and can therefore be easily purified to a high purity by recrystallization or like processes. By mixing such high-purity organic salts, the room-temperature molten salt of the present invention can be easily obtained in high purity.

The room-temperature molten salt of the present invention has a remarkably lower solidifying point and retains its liquid state over a wider temperature range than single-component room-temperature molten salts, and therefore shows promise for a wide variety of applications. Moreover, unlike single-component room-temperature molten salts, the room-temperature molten salt of the present invention can be given various properties suitable for various applications by appropriately selecting the types and proportions of the starting organic salts.

Due to the above features, the room-temperature molten salt of the present invention can be suitably used for nonaqueous battery electrolytes or electrolytic solutions, which are required to have high purity. Further, the room-temperature molten salt of the present invention has a very low solidifying point, and thus can provide a battery with excellent low-temperature properties.

The room-temperature molten salt of the present invention can also be used as a solvent for various organic synthesis reactions and as an extraction solvent for separation and purification in organic synthesis.

Further, since the room-temperature molten salt of the present invention has high heat resistance, is liquid over a wide temperature range and has high ionic conductivity, it can also be used as an electrolytic solution for various plating processes.

The room-temperature molten salt of the present invention does not undergo a phase change until it reaches a low temperature and has excellent low temperature properties. Because of these features, the room-temperature molten salt can be used as an electrolyte and/or electrolytic solution for fuel cells, dye-sensitized solar cells, biological batteries or capacitors, an electro-rheological fluid, a heat storage medium, a catalyst, etc.

1. A room-temperature molten salt comprising a mixture of two or more organic salts with different anionic moieties and different organic cationic moieties, the room-temperature molten salt having a solidifying point lower than that of any of the individual organic salts.

2. A room-temperature molten salt according to claim 1, wherein the two or more organic salts are selected from the group consisting of the organic salts represented by formulae (I), (II), (Im) and (IV): 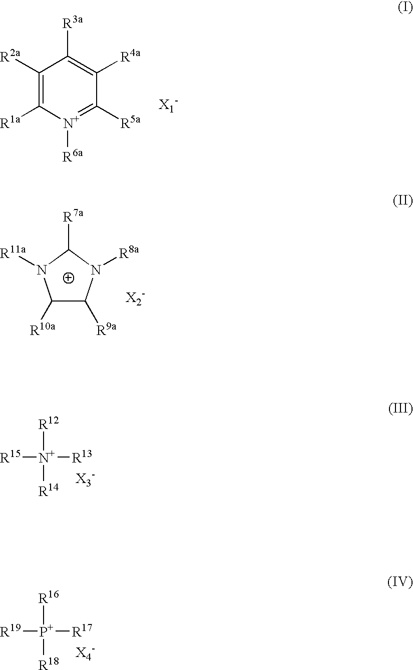
wherein R^{1a} to R^{5a}, R^{7a}, R^{9a} and R^{10a} are the same or different and each represents a hydrogen atom, a halogen atom, an alkyl group, a cycloalkyl group, a heterocyclic group, a haloalkyl group, an aralkyl group, an aryl group, an alkoxy group, an aryloxy group or an aralkyloxy group; R^{8a} is a hydrogen atom, an alkyl group, a cycloalkyl group, a heterocyclic group, a haloalkyl group, an aralkyl group or an aryl group; R^{6a}, R^{11a}, R¹², R¹³, R¹⁴, R¹⁵, R¹⁶, R¹⁷, R¹⁸ and R¹⁹ are the same or different and each represents an alkyl group, a cycloalkyl group, a heterocyclic group, a haloalkyl group, an aralkyl group or an aryl group; two groups selected from R¹², R¹³, R¹⁴ and R¹⁵ may be linked at their ends to form, together with the adjacent nitrogen atom, a nitrogen-containing aliphatic heterocycle; two groups selected from R¹⁶, R¹⁷, R¹⁸ and R¹⁹ may be linked at their ends to form, together with the adjacent phosphorus atom, aphosphorus-containing aliphatic heterocycle; and X₁⁻, X₂⁻, X₃⁻ and X₄⁻ are each a conjugate base of a Brönsted acid.

3. A room-temperature molten salt according to claim 1 or 2, wherein at least one of the two or more organic salts is a solid at room temperature.

4. A room-temperature molten salt according to claim 1 or 2, wherein all of the two or more organic salts are solids at room temperature.

5. A room-temperature molten salt according to claim 1 or 2, wherein at least one of the two or more organic salts is selected from the group consisting of the organic salts represented by formulae (V) and (VI): 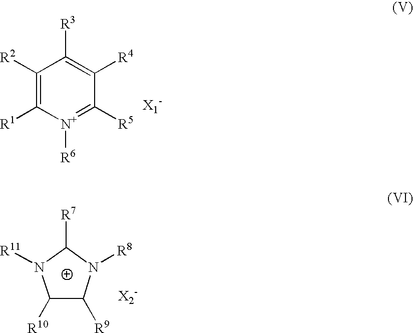
wherein R¹ to R⁵, R⁷, R⁹ and R¹⁰ are the same or different and each represents a hydrogen atom, a halogen atom, an alkyl group, a cycloalkyl group, a heterocyclic group, a haloalkyl group, an aralkyl group, an aryl group, an alkoxy group, an aryloxy group or an aralkyloxy group; R⁸ is a hydrogen atom, an alkyl group, a cycloalkyl group, a heterocyclic group, a haloalkyl group, an aralkyl group or an aryl group; R⁶ and R¹¹ are the same or different and each represents a C₁₋₁₀ alkyl group in which at least one hydrogen atom is substituted by fluorine; and X₁⁻ and X₂⁻ are each a conjugate base of a Brönsted acid.

6. A room-temperature molten salt according to claim 5, wherein all of the two or more organic salts are selected from the group consisting of the organic salts represented by formulae (V) and (VI).

7. A room-temperature molten salt according to claim 5, wherein at least one of the two or more organic salts is a solid at room temperature.

8. A room-temperature molten salt according to claim 5, wherein all of the two or more organic salts are solids at room temperature.

9. A room-temperature molten salt according to claim 5, wherein, in formulae (V) and (VI), R¹ to R⁵, R⁷, R⁹ and R¹⁰ are the same or different and each represents a hydrogen atom, a halogen atom, an alkyl group or a haloalkyl group; R⁸ is an alkyl group; R⁶ and R¹¹ are the same or different and each represents a group of the formula —CH₂R¹² wherein R¹² is a straight- or branched-chain C, -alkyl group in which at least one hydrogen atom is substituted by fluorine.

10. A room-temperature molten salt according to claim 6, wherein all of the two or more organic salts are selected from the group consisting of the organic salts represented by formula (V) and are solids at room temperature.

11. A room-temperature molten salt according to claim 6, wherein all of the two or more organic salts are selected from the group consisting of the organic salts represented by formula (VI) and are solids at room temperature.

12. A room-temperature molten salt according to claim 6, wherein the two or more organic salts are at least one organic salt that is selected from the group consisting of the organic salts represented by formula (V) and is a solid at room temperature, and at least one organic salt that is selected from the group consisting of the organic salts represented by formula (VI) and is solid at room temperature.

13. A room-temperature molten salt according to claim 6, wherein the two or more organic salts are two organic salts that are selected from the group consisting of the organic salts represented by formulae (V) and (VI) and that are solids at room temperature; one of the organic salts having an anionic moiety represented by the formula
(RfSO₂)₂N⁻ or (RfSO₂)(Rf′SO₂)N⁻
wherein Rf and Rf′ are different and each represents a polyfluoroalkyl group; and
the other of the organic salts having an anionic moiety represented by the formula
Rf′SO₃⁻
wherein Rf′ is a polyfluoroalkyl group.

14. A room-temperature molten salt obtainable by mixing two or more organic salts with different anionic moieties and different organic cationic moieties, the room-temperature molten salt having a solidifying point lower than that of any of the individual organic salts.

15. A process for producing a room-temperature molten salt, comprising mixing two or more organic salts with different anionic moieties and different organic cationic moieties, the room-temperature molten salt having a solidifying point lower than that of any of the individual organic salts.

16. A process according to claim 15, wherein the two or more organic salts are selected from the group consisting of the organic salts represented by formulae (I) to (IV).

17. A process according to claim 15 or 16, wherein at least one of the two or more organic salts is a solid at room temperature.

18. A process according to claim 15 or 16, wherein all of the two or more organic salts are solids at room temperature.

19. A process according to claim 15, wherein the two or more organic salts are selected from the group consisting of the organic salts represented by formulae (V) and (VI) and are solids at room temperature.

20. An electrolytic solution comprising a room-temperature molten salt according to claim 1 or claim 14.

21. A battery comprising an electrolytic solution according to claim 20, a positive electrode, a negative electrode and a separator.

22. A battery according to claim 21, which is a nonaqueous lithium secondary battery.

23. A solvent for use in organic reaction solvent comprising a room-temperature molten salt according to claim 1 or claim 14.

24. An extraction solvent comprising a room-temperature molten salt according to claim 1 or claim 14.

25. A capacitor comprising an electrolyte or electrolytic solution that comprises a room-temperature molten salt according to claim 1 or claim 14.

26. An electric double layer capacitor comprising an electrolyte or electrolytic solution that comprises a room-temperature molten salt according to claim 1 or claim 14.

27. A dye-sensitized solar cell comprising a room-temperature molten salt according to claim 1 or claim 14.

28. A fuel cell comprising a room-temperature molten salt according to claim 1 or claim 14.

29. A polymer electrolyte fuel cell comprising a room-temperature molten salt according to claim 1 or claim 14.
